(19) United States
(12) Patent Application Publication
(10) Pub. No.: US 2019/0313709 A1
(43) Pub. Date: Oct. 17, 2019

(54) ASYMMETRIC BRASSIERE FOR USE WITH A PROSTHETIC BREAST

(71) Applicant: Gerri Hollins, Louisville, KY (US)

(72) Inventor: Gerri Hollins, Louisville, KY (US)

(21) Appl. No.: 16/256,546

(22) Filed: Jan. 24, 2019

Related U.S. Application Data
(60) Provisional application No. 62/657,783, filed on Apr. 14, 2018.

(51) Int. Cl. A41C 3/14; A61F 2/52

(52) U.S. Cl. CPC A41C 3/148; A41C 3/02; A61F 2/52

(57) ABSTRACT

An asymmetric brassiere for use with a prosthetic breast is disclosed. The asymmetric brassiere provides a front closure, which provides greater support for the front of the user's body, and enables fastening without shifting the position of the prosthetic. Further, a conductive fabric is provided which cups under the prosthetic. The asymmetric brassiere provides a user's pre-surgery appearance and equilibrium at a reasonable cost.

### RELATED APPLICATION(S)

Under provisions of 35 U.S.C. § 119e, the Applicant(s) claim the benefit of U.S. provisional application No. 62/657,783 filed Apr. 14, 2018, which is incorporated herein by reference.

### FIELD OF THE INVENTION

The present invention relates generally to health care devices and accessories, and in particular to an asymmetric brassiere for use with a prosthetic breast.

### BACKGROUND OF THE INVENTION

For millions of breast cancer survivors, restoring the pre-surgery appearance and equilibrium is essential for their emotional well-being. However, most breast cancer survivors are unable to afford reconstructive surgery with a breast implant.

A search of the prior art reveals various devices which have been developed to address this set of problems, or related problems. None are closely related to the present invention, but several include features which resemble those of the present invention. Each has proven to be less than satisfactory for the present purpose in its own way.

The U.S. Pat. No. 5,180,326 (priority Jan. 26, 1992), titled “Reversible mastectomy brassiere” provides a reversible mastectomy brassiere which may be worn soon after surgery is disclosed with the primary purpose of this brassiere concept being to provide a mastectomy brassiere which may position a prosthesis on the left breast, or when said brassiere is completely reversed, to position a prosthesis on the right breast of the mastectomy patient. This design further includes front fasteners which function equally well irrespective of the side on which the brassiere is worn. The brassiere will provide adequate coverage for the patient soon after surgery, without the necessity of an expensive and time consuming special fitting. The second important aspect of this design is its continuous unbroken and smooth appearance when worn because the brassiere configuration and all outer edges have no apparent sewing or standard finishing of any kind on the outer surface of the brassiere. This design blends smoothly with the body's contour and gives visual reassurance and psychological relief to the patient recovering from the trauma of surgery. However, the disclosed mastectomy brassiere may not provide the required flexibility to adjust or change a prosthesis. Further, the disclosed brassiere may not be able to restore the pre-surgery appearance and equilibrium.

The U.S. Pat. No. 5,158,541 (priority Jan. 23, 1992) titled “Mastectomy compression surgical brassiere” provides a mastectomy compression surgical brassiere having as a component a mastectomy brassiere having a pair of camisole shoulder straps and lined brassiere cups, a rectangular compression pad sewn to the inside of the front of the brassiere at substantially its mid-point, the rectangular compression pad is covered with a soft porous VELCRO pile looped structure on its external surface lined with a soft fabric, and has an elastic return force band having a portion of a VELCRO hook fabric fastener fabric secured on the side of said return force band away from the body of said compression pad adapted to engage the loops of the VELCRO pile structure cover on said compression pad to retain surgical gauze pads against the incision between the body and the compression pad, and wherein a loop fabric type hook and fastener fabric is secured to the elastic return force band positioned to attach the VELCRO hooks on the return force band to said brassiere to retain the compression pad of the brassiere and surgical dressing pads firmly in place against a mastectomy incision, and to maintain the brassiere firmly against the body of the wearer. However, the disclosed mastectomy compression surgical brassiere may not be able to restore the pre-surgery appearance and equilibrium.

The U.S. Pat. No. 3,447,538 (priority Oct. 7, 1966), titled “Mastectomy brassieres and garments for same” provides a garment formed of soft material for the use of patients who have experienced a single mastectomy or a second or a complete mastectomy. The innermost element is in the nature of an undershirt to the front face of which brassiere elements are secured. The rear face fits the patients hack snugly as an undershirt. Secured to the front of this first element is a layer of goods in the nature of a swing on which is mounted the false breastforms attached only at the shoulder, side and arm seams. The outer element or false front concealing the breastforms and brassiere elements is sewed to the above named first element on all sides except the bottom. However, the disclosed garment may not be able to restore the pre-surgery appearance and equilibrium.

The U.S. Pat. No. 3,701,168 (priority Aug. 23, 1971), titled “Brassieres for mastectomy patients” provides a brassiere which comprises a breast pocket or cup, which may be made of any convenient material or form, but has a top panel and a bottom panel stitched to the top panel in order to provide uplifting support for the remaining breast. Both panels are preferably made from cotton material. The inner side edge of the pocket comprises a featherbone strip to prevent that side of the pocket from collapsing. The outer side edge of the pocket comprises a hem, backed with a featherbone strip. However, the disclosed brassiere may not be able to restore the pre-surgery appearance and equilibrium.

The U.S. Pat. No. 3,094,125 (priority Apr. 28, 1961), titled “Mastectomy brassiere” provides a mastectomy garment which includes an underbra, a prosthetic breast pad, and an outerbra. The underbra has a supporting cup for the normal breast and a substantially flat band portion to fit to the chest of the removed breast, enabling a firm, flat-fitting garment over the remaining chest surface of the removed breast. The prosthesis or false breast pad is removably adhered to the outer surface of the flat, binding portion. The outerbra, such as a conventional off-the-shelf brassiere, that overlies and is secured to the underbra forming a composite unitary prosthetic brassiere with the prosthetic breast pad positioned therebetween and means for adjustment of the brassiere to the wearer and individual adjustment of the prosthetic breast pad. However, the disclosed brassiere may not be able to restore the pre-surgery appearance and equilibrium.

The U.S. Pat. No. 4,637,398 (priority Oct. 11, 1985), titled “Mastectomy brassiere” provides a mastectomy garment includes an underbra, a breast prosthesis, and an outerbra. The underbra is of lightweight garment foundation material having a cup for the normal breast and a flat band portion for the removed breast, enabling a firm, flat-fitting garment over the remaining chest surface of the removed breast. The prosthesis or false breast itself is removably adhered to the outer surface of the flat, binding portion. The outerbra such as a conventional off-the-shelf brassiere is placed over the underbra and the adhering prosthesis, the underbra and outerbra are then secured together to prevent relative movement between them. However, the disclosed brassiere may not be able to restore the pre-surgery appearance and equilibrium.

The U.S. Patent Appl. No. 2002/0102913A1 (priority Jan. 31, 2001), titled “Mastectomy brassiere” provides a mastectomy brassiere, comprising: a member adapted to encircle a torso; a first cup on the member adapted to support a reconstructed breast having an unnaturally firm implant, the first cup being adjustable to comform in shape at least in part to the reconstructed breast; and, a second cup on the member adapted to support a natural breast, the second cup having padding for urging the natural breast to conform in appearance to the reconstructed breast with the unnaturally firm implant, whereby the brassiere can be custom tailored to support natural and reconstructed breasts differing significantly from one another in firmness, yet impart to the natural and reconstructed breasts an appearance of similar firmness. The brassiere can further comprise: a deformable support wire for adjusting the partial perimeter of the first cup to conform to the partial perimeter of the reconstructed breast; and, padding in the second cup for urging the natural breast to conform in appearance to the reconstructed breast. A method for fitting a brassiere to mastectomy patients is also disclosed. However, the disclosed brassiere may not be able to restore the pre-surgery appearance and equilibrium.

The U.S. Pat. No. 2,779,943 (priority Nov. 22, 1954), titled “Post-mastectomy brassiere” provides an attachment for a brassiere having a body-encircling band, shoulder straps and a pair of breast-receiving cups, to adapt the brassiere for use by a post-masters to my patient; said attachment comprising a body of soft fabric having a first portion attachable to the inner side of said band so as to span one of said cups and retain therein artificial breast form; said body having a second portion joined to one side of said first portion and extended laterally therefrom for attachment at its free end to said band at a point between the other cup and the end of the band adjacent the other cop; said second portion being shaped to extend beneath the lower edge of said other cup; said body having a third portion joined to the lower part of said first portion and extending downwardly therefrom below said band; an elastic strap on the lower end of said third portion; and means on the lower end of said strap for attaching said strap to a garment of the wearer. However, the disclosed brassiere may not be able to restore the pre-surgery appearance and equilibrium.

Accordingly, there is a need for an improved less-expensive brassiere to restore the pre-surgery appearance and equilibrium that may also overcome one or more of the abovementioned problems and/or limitations.

### SUMMARY OF THE INVENTION

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

Accordingly, the present disclosure is directed to an asymmetric brassiere for use with a prosthetic breast. The brassiere provides a front closure, which provides greater support for the front of the user's body, and enables fastening without shifting the position of the prosthetic. A conductive fabric is provided which cups under the prosthetic. The brassiere provides a user's pre-surgery appearance and equilibrium at a reasonable cost.

The disclosed asymmetric brassiere is a simple and inexpensive device which enables the user to secure a prosthetic breast. Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a front view of an asymmetric brassiere, in accordance with a first exemplary embodiment.

FIG. 2 is a top view of the asymmetric brassiere of FIG. 1.

FIG. 3 is a right side view of the asymmetric brassiere of FIG. 1.

FIG. 4 is a left side view of the asymmetric brassiere of FIG. 1.

FIG. 5 is a top right-side perspective view of the asymmetric brassiere of FIG. 1.

FIG. 6 is a top left-side perspective view of the asymmetric brassiere of FIG. 1.

### DETAILED DESCRIPTION OF THE INVENTION

As a preliminary matter, it will readily be understood by one having ordinary skill in the relevant art that the present disclosure has broad utility and application.

As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise.

Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of an asymmetric brassiere, embodiments of the present disclosure are not limited to use only in this context.

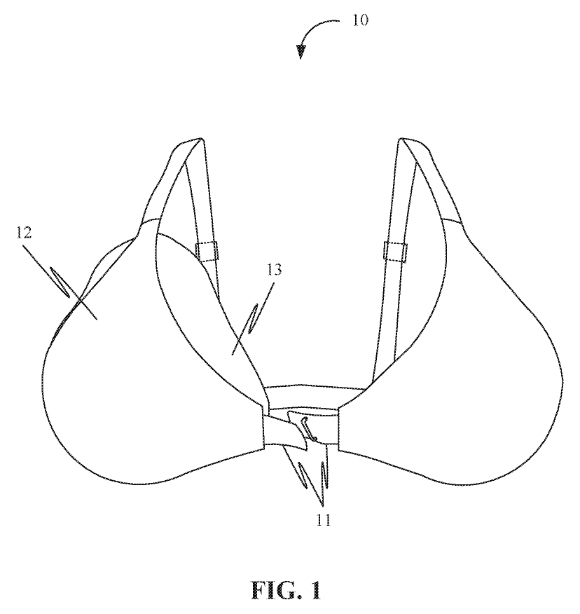

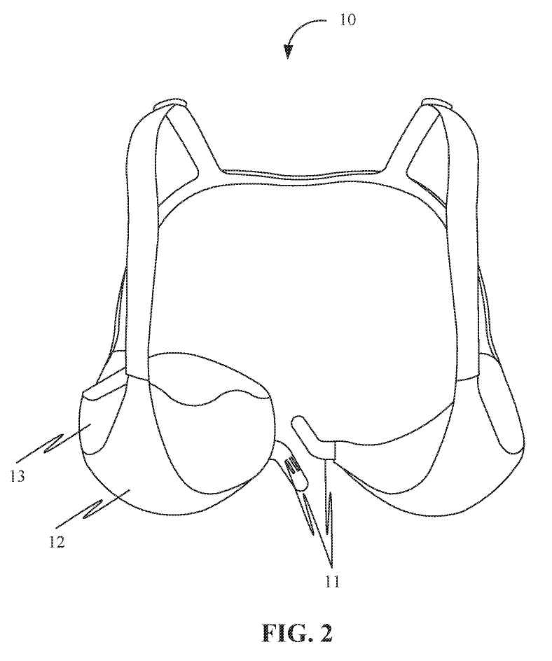

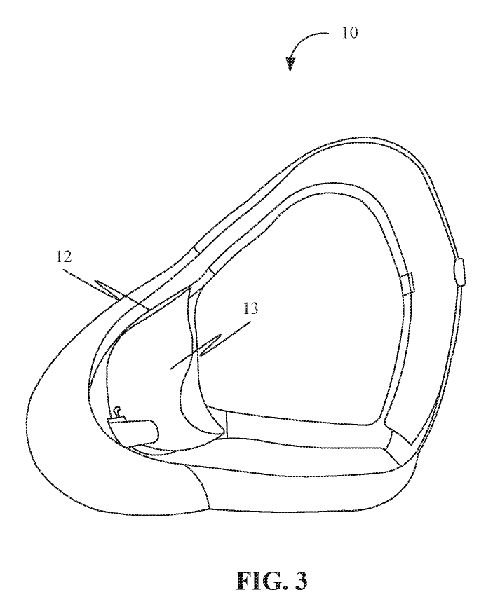

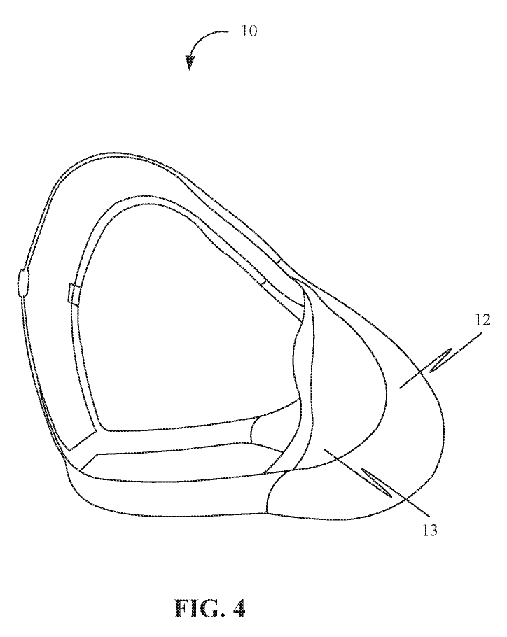

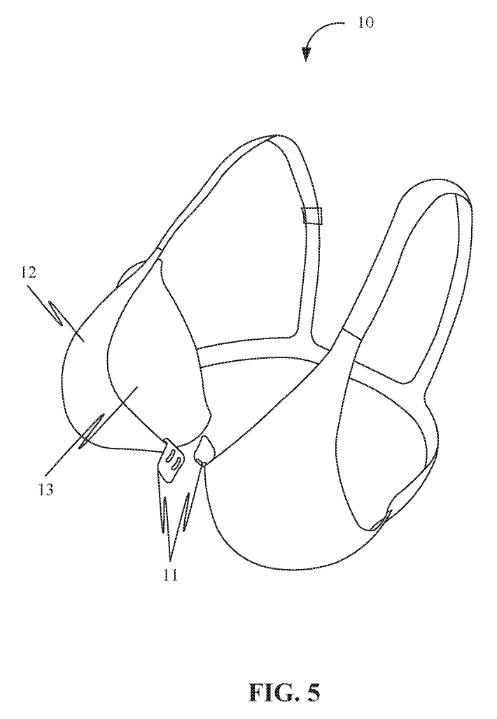

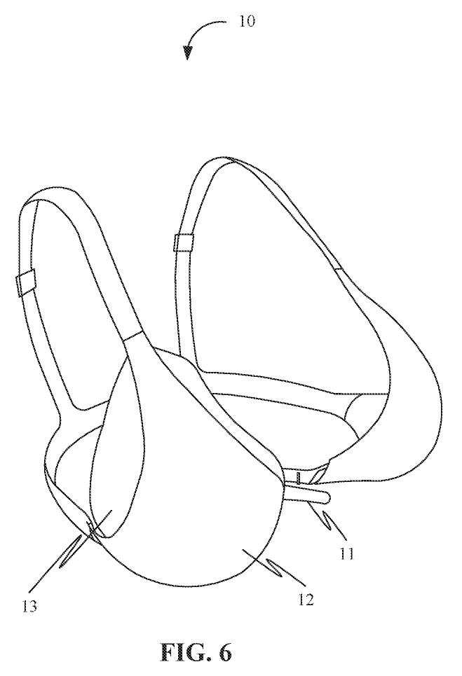

FIG. 1 is a front view of an asymmetric brassiere 10, in accordance with a first exemplary embodiment. The asymmetric brassiere 10 includes one or more of a fastener 11, a prosthetic cup 12, and a prosthetic breast 13. FIG. 2 is a top view of the asymmetric brassiere 10. FIG. 3 is a right side view of the asymmetric brassiere 10. FIG. 4 is a left side view of the asymmetric brassiere 10. FIG. 5 is a top right-side perspective view of the asymmetric brassiere 10. FIG. 6 is a top left-side perspective view of the asymmetric brassiere 10.

The asymmetric brassiere 10 is intended for use with the prosthetic breast 13 supplied by the user. The asymmetric brassiere 10 includes the front fastener 11, which provides greater support for the front of the user's body, and enables fastening without shifting the position of the prosthetic breast 13. The fastener 11 provides front closure of the asymmetric brassiere 10.

The prosthetic cup 12 is provided for the prosthetic breast 13. The prosthetic cup 12 may be manufactured from a conductive fabric which cups under the prosthetic breast 13. Further, different versions of the asymmetric brassiere 10 may be provided with the prosthetic cup 12 on the left side, the right side, or both sides. The asymmetric brassiere 10 may be provided in a full range of sizes and colors.

In order to use the asymmetric brassiere 10, the user may snap open the fastener 11 and put on the asymmetric brassiere 10, position the prosthetic breast 13 comfortably within the prosthetic cup 12, and close the fastener 11.

Further, the asymmetric brassiere 10 may be manufactured from flexible, durable materials which are easily cleaned, such as cotton fabric or a cotton/polyester blend. The fastener 11 may be manufactured from a rigid, durable material, such as steel, plastic, or aluminum alloy.

Components and methods listed above are preferable, but artisans will recognize that alternate components and methods could be selected without altering the scope of the invention.

Although the invention has been explained in relation to its preferred embodiment, it is to be understood that many other possible modifications and variations can be made without departing from the spirit and scope of the invention.

I claim:

1. An asymmetric brassiere which is intended for use with a prosthetic breast supplied by the user, providing a front closure with a fastener, and a prosthetic cup for the prosthetic breast.

2. The asymmetric brassiere of claim 1, wherein the prosthetic cup is manufactured from a conductive fabric which cups under the prosthetic breast.

3. The asymmetric brassiere of claim 1, wherein different versions of the asymmetric brassiere are provided with a prosthetic cup on the left side, the right side, or both sides.

4. The asymmetric brassiere of claim 1, wherein the user may snap open the fastener and put on the asymmetric brassiere, position the prosthetic breast comfortably within the prosthetic cup, and close the fastener.

5. The asymmetric brassiere of claim 1, wherein the asymmetric brassiere is manufactured from flexible, durable materials which are easily cleaned, such as cotton fabric or a cotton/polyester blend.

6. The asymmetric brassiere of claim 1, wherein the fastener is manufactured from a rigid, durable material, such as steel, plastic, or aluminum alloy.
